(19) United States
(12) Patent Application Publication
(10) Pub. No.: US 2026/0182961 A1
(43) Pub. Date: Jul. 2, 2026

(54) MULTIFREQUENCY ULTRASOUND MEASURING SYSTEMS AND METHODS

(71) Applicant: Provisio Medical, Inc., San Diego, CA (US)

(72) Inventors: Stephen Eric Ryan, San Diego, CA (US); Nestor E. Cabrera-Munoz, San Diego, CA (US)

(21) Appl. No.: 19/129,873

(22) PCT Filed: Nov. 16, 2023
(86) PCT No.: PCT/US2023/080137

§ 371 Date: May 14, 2025

Related U.S. Application Data
(60) Provisional application No. 63/384,763, filed on Nov. 22, 2022.

(51) Int. Cl. A61B 8/00; A61B 8/08; A61B 8/12

(52) U.S. Cl. CPC A61B 8/445; A61B 8/0891; A61B 8/12; A61B 8/4494; A61B 8/5223

(57) ABSTRACT

An ultrasound probe including a flexible body elongated along a longitudinal axis and assembled for insertion into a structure. The ultrasound probe has a plurality of ultrasound transducers arranged along the flexible body and a shared signal conductor shared among a first and second of the plurality of ultrasound transducers. The first transducer is configured to respond to a first ultrasound frequency range and the second transducer configured to respond to a second ultrasound frequency range different from the first ultrasound frequency range.

### REFERENCE TO RELATED APPLICATIONS

This application claims the benefit of U.S. Provisional Application No. 63/384763, filed Nov. 22, 2022, which is hereby incorporated by reference in its entirety.

### BACKGROUND

### Field of the Disclosure

The present disclosure relates generally to systems, methods, and devices that utilize ultrasound to gather dimensional and physiological information about structures such as fluid-filled body vessels.

### Description of Related Art

Obtaining and utilizing structural information about patients is a critical aspect of diagnosing and treating many medical conditions. For example, within the field of endovascular medicine, it is important to gain structural and physiological information about diseased blood vessels when selecting among interventional techniques such as angioplasty, stents, and/or surgery. Recent studies have illustrated that the predominate cause of endovascular treatment failure is inaccurate sizing of vessels or inadequate treatment to achieve the lumen dimensions desired over an entire stenotic lesion. An improperly selected, dimensioned, and/or positioned medical device (e.g., a stent) and/or treatment can lead to highly adverse outcomes including avoidable death. Typical techniques used for analyzing the structural features of blood vessels include angiography. However, angiography only provides limited and imprecise information about the size and morphology of blood vessels and often does not allow the physician to adequately assess the lesion prior to treatment. Recent studies have shown that outcomes are significantly improved through the use of more advanced, more accurate imaging techniques.

Some imaging catheters utilize ultrasound or optical technologies to provide a more accurate cross-sectional imaging that may then be interpreted by the physician to determine, among other characteristics, the dimensions of the lumen surrounding the catheter. For example, Intravascular Ultrasound (IVUS) and Optical Coherence Tomography (OCT) have been used in interventional diagnostic procedures to image blood vessels to locate and characterize atherosclerosis and other vessel diseases and defects.

IVUS and OCT images can be used to determine information about a vessel, including vessel dimensions, and is typically much more detailed than the information that is obtainable from traditional angiography images, which are generally limited to two-dimensional shadow images of the vessel lumen. The information gained from more accurate imaging techniques can be used to better assess physiological conditions, select particular procedures, and/or improve performance of the procedure. Some systems are described in which multiple lumen wall distances are measured and a shape of the wall is calculated using the distance measurements such as described in U.S. Pat. No. 10,231,701 filed Mar. 14, 2014 (the '701 Patent), the entire contents of which is herein incorporated by reference.

While current IVUS and OCT systems provide additional and more detailed information compared to angiograms, these IVUS and OCT systems introduce significant additional time, cost and complexity into minimally-invasive procedures. The components of these systems (e.g., transducers, wires, imaging circuitry, fiber-optics, etc.) can occupy a large footprint within the blood vessel and must often be deployed independently and at separate times from interventional procedures (e.g., angioplasty). Further, the images produced by IVUS and OCT systems may not directly provide useful information about blood vessels and are typically subject to nonconforming interpretations of different physicians. Thus, there is a need for an improved and more efficient way to get needed information about a vessel or structure, particularly information about the diameter and multi-dimensional profile of a vessel or structure, while not sacrificing speed and footprint needed for timely, efficient, and effective treatment.

### SUMMARY

Embodiments of the present disclosure include a novel implementation of an ultrasound probe using differentiated transducers to approximate the dimensions of fluid-filled structures. Some embodiments include an elongated flexible body such as a catheter with multiple ultrasound transducers arranged circumferentially about the catheter for generating and receiving ultrasound signals to and from surrounding structure. The signals are delivered via a shared conductor to a programmable device used to analyze and transform the signals into distance measurements between the flexible body and surrounding structure (e.g., a vessel wall). The transducers are configured to selectively respond to different ranges frequencies of ultrasound signals and to generate electrical excitation pulses representing the different frequencies. The excitation pulses are delivered through the shared conductor to the programmable device, which is programmed to associate the signals representing particular ranges of ultrasound frequencies to the different transducers of the body. Based on the associated signals, the device is further programmed to calculate physical distances between various points of the flexible body to the surrounding structure. These distance measurements may then be used to calculate other dimensional characteristics of the structure (e.g., diameter, morphology, and other features). In some embodiments, the flexible body is moved through a structure as these distance measurements are obtained and used to provide dimensional characteristics along a longitudinal extent of the structure. Utilizing these measurements, some embodiments approximate for the physician the shape and size of the structure into which the elongated body is placed and permit them to use this information to perform therapeutic procedures with tools connected to the conduit (e.g., an angioplasty balloon) while in place within the vessel.

For purposes of summarizing the disclosure and the advantages achieved over the prior art, certain objects and advantages of the disclosure are described herein. Not all such objects or advantages may be achieved in any particular embodiment. Thus, for example, those skilled in the art will recognize that the invention may be embodied or carried out in a manner that achieves or optimizes one advantage or group of advantages as taught herein without necessarily achieving other objects or advantages as may be taught or suggested herein.

In a first aspect, an ultrasound probe is provided. The ultrasound probe includes a flexible body elongated along a longitudinal axis and assembled for insertion into a structure, a plurality of ultrasound transducers arranged along the flexible body, and a shared signal conductor shared among a first and second of the plurality of ultrasound transducers, the first transducer configured to respond to a first ultrasound frequency range and the second transducer configured to respond to a second ultrasound frequency range different from the first ultrasound frequency range.

In some embodiment, the first frequency range is between about 20 and 35 MHz and the second frequency range is between about 35 and 50 MHz. In some embodiments, a resonant frequency difference between the first and second transducer is at least about 10 MHz. In some embodiments, the first transducer includes a first piezoelectric layer having dimensions configured to cause the first layer to resonate within the first ultrasound frequency range and the second transducer includes a second piezoelectric layer having dimensions configured to cause the second layer to resonate within the second ultrasound frequency range. In some embodiments, a piezoelectric layer of the first ultrasound transducer has a thickness of between about 60 and 100 microns and a resonant frequency of between about 20 and 35 MHz and where a piezoelectric layer of the second transducer has a thickness between about 40 and 60 microns and a resonant frequency of between about 35 and 50 MHz. In some embodiments, neither of the first and second transducers include a conductive matching layer. In some embodiments, a conductive electrode is layered over the first and second transducers. In some embodiments, at least one of the first and second transducers does not include a matching layer of a thickness that is equal to or greater than about a quarter ultrasound wavelength corresponding to the respective first and second frequency ranges. In some embodiments, the plurality of ultrasound transducers are arranged circumferentially about the flexible body. In some embodiments, the first ultrasound transducer and second ultrasound transducer are circumferentially adjacent to each other among the plurality of ultrasound transducers. In some embodiments, the third and fourth ultrasound transducers of the plurality of transducers that share a conductor, the third and fourth ultrasound transducers arranged circumferentially across from the first and second ultrasound transducers, respectively. In some embodiments, a therapeutic device is arranged at a predetermined location with respect to the plurality of transducers. In some embodiments, the therapeutic device includes an angioplasty balloon. In some embodiments, one or more of the plurality of transducers are arranged within the angioplasty balloon.

In another aspect an ultrasound system for measuring the dimensions of a structure is provided. The ultrasound system includes a flexible body elongated along a longitudinal axis and assembled for insertion into the structure, a plurality of ultrasound transducers arranged circumferentially about the flexible body, a shared signal conductor shared among a first and a second ultrasound transducer of the plurality of ultrasound transducers, the shared conductor arranged to transmit electrical signals from the first and second ultrasound transducers, the first transducer configured to respond to a first ultrasound frequency range and the second transducer configured to respond to a second ultrasound frequency range different from the first ultrasound frequency range, and one or more processors programmed and configured to calculate a plurality of distances between the elongate flexible body and an inner wall of the structure, the calculating based on the electrical signals transmitted from the plurality of ultrasound transducers.

In some embodiments, the one or more processors are further configured to calculate cross-sectional dimensions and shapes of the structure based on the plurality of distances. In some embodiments, the first frequency range is between about 20 and 30 MHZ and the second frequency range is between about 30 and 50 MHz. In some embodiments, the one or more processors are programmed and configured to transmit a first set of signals through the shared conductor to cause the first transducer to transmit ultrasound signals of the first frequency range toward the structure, obtain and process ultrasound signals responsive to the first set of signals, after obtaining the ultrasound signals responsive to the first set of signals, transmit a second set of signals through the shared conductor to cause the second transducer to transmit ultrasound signals of the second frequency range toward the structure, and obtain and process ultrasound signals responsive to the second set of signals, where the calculating a plurality of distances between the elongate flexible body and an inner wall of the structure is based on analyzing the processed signals responsive to the first and second sets of signals. In some embodiments, analyzing the processed signals includes identifying characteristics of the medium between the elongate flexible body and structure. In some embodiments, identifying the characteristics includes identifying movement of the medium. In some embodiments, the medium is blood and the structure is a blood vessel. In some embodiments, one or more processors are programed to isolate the response signal from an activated transducer having a lower frequency. In some embodiments, the system further includes a flexible expandable balloon arranged about the flexible body, where one or more of the plurality of transducers are arranged within the expandable balloon, and where calculating a plurality of distances between the elongate flexible body and an inner wall of the structure includes calculating distances between the elongate flexible body and an inner wall of the expandable balloon.

In another aspect, a method for measuring the dimensions of a structure using an ultrasound probe is provided, The method includes generating ultrasound signals of a first frequency range from a first subset of a plurality of ultrasound transducers sharing a signal conductor, obtaining ultrasound signals responsive to the signals of the first frequency range, generating ultrasound signals of a second frequency from a second subset of a plurality of ultrasound transducers sharing the signal conductor, the second frequency range different from the first frequency range, obtaining ultrasound signals responsive to the signals of the second frequency range, and analyzing the obtained ultrasound signals responsive to the signals of the first and second frequency ranges and generating an ultrasound image based on the analyzing.

In some embodiments, the first subset of transducers are selectively responsive to the first frequency range and the second subset of transducers are selectively responsive to the second frequency range, where the second subset of transducers are substantially unresponsive to the first frequency range and the first subset of transducers are substantially unresponsive to the second frequency range. In some embodiments, the first frequency range is between about 20 and 30 MHz and the second frequency range is between about 30 and 50 MHz. In some embodiments, the first and second frequency ranges are separated by at least about 10 MHz. In some embodiments, the method includes calculating dimensions and shapes of the structure based on the ultrasound image. In some embodiments, the structure is a blood vessel and the ultrasound probe is placed within the blood vessel while obtaining ultrasound signals responsive to the signals of the first and second frequency ranges. In some embodiments, the ultrasound probe includes a therapy-delivery element and the method further includes generating time-sequenced ultrasound images, and positioning the therapy-delivery element within the blood vessel based on the time-sequenced ultrasound images. In some embodiments, the therapy-delivery element is an angioplasty balloon. In some embodiments, obtaining ultrasound signals responsive to the signals of the first or second frequency range include obtaining signals echoed from the angioplasty balloon and where the method further includes determining an amount of expansion of the angioplasty balloon based on analyzing the obtained signals.

### BRIEF DESCRIPTION OF THE DRAWINGS

Embodiments of the disclosure will be described hereafter in detail with particular reference to the drawings. Throughout this description, like elements, in whatever embodiment described, refer to common elements wherever referred to and reference by the same reference number. The characteristics, attributes, functions, interrelations ascribed to a particular element in one location apply to that element when referred to by the same reference number in another location unless specifically stated otherwise. In addition, the exact dimensions and dimensional proportions to conform to specific force, weight, strength and similar requirements will be within the skill of the art after the following description has been read and understood.

All figures are drawn for ease of explanation of the basic teachings of the present disclosure only; the extensions of the figures with respect to number, position, relationship and dimensions of the parts to form examples of the various embodiments will be explained or will be within the skill of the art after the present disclosure has been read and understood.

FIG. 1 is an illustrative diagram of an ultrasound catheter probe system according to some embodiments.

FIG. 2A is an illustrative side perspective diagram of an ultrasound catheter probe placed within a lumen according to some embodiments.

FIG. 2B is a cross-sectional perspective diagram of the ultrasound catheter probe of FIG. 2A.

FIG. 3A is an illustrative diagram of a transducer with a dedicated conductor.

FIG. 3B is an illustrative diagram of multiple transducers sharing a conductor according to some embodiments.

FIG. 4A is an illustrative diagram of a piezoelectric transducer.

FIG. 4B is an illustrative diagram of a transducer configured for a selective response to a frequency range according to some embodiments.

FIG. 4C is an illustrative diagram of a transducer configured for selectively responding to another frequency range according to some embodiments.

FIG. 5 is an illustrative chart of exemplary frequency responses of transducers sharing a conductor according to some embodiments.

FIG. 6A is an illustrative chart of a modulated electric pulse for activating a transducer according to some embodiments.

FIG. 6B is an illustrative chart of an intensity-envelope of a modulated electric pulse for activating a transducer according to some embodiments.

FIG. 6C is an illustrative chart of a frequency-and intensity-modulated electric pulse for activating a transducer according to some embodiments.

FIG. 7A is an illustrative chart of a frequency response from multiple transducers sharing a conductor.

FIG. 7B is another illustrative chart of a frequency response from multiple transducers sharing a conductor.

FIG. 8 is an illustrative diagram of an array of transducers sharing conductors according to some embodiments.

FIG. 9A is an illustrative diagram of an ultrasound catheter having shared conductors according to some embodiments.

FIG. 9B is an illustrative diagram of a transducer arrangement with shared conductors according to some embodiments.

FIG. 10 is a block diagram of a process for generating an ultrasound image using transducers sharing a conductor according to some embodiments.

FIG. 11 is a block diagram of a process for using an imaging probe with transducers sharing a conductor to calculate distance measurements according to some embodiments.

### DETAILED DESCRIPTION

In order that embodiments of the disclosure may be clearly understood and readily carried into effect, certain embodiments of the disclosure will now be described in further detail with reference to the accompanying drawings. The description of these embodiments is given by way of example only and not to limit the scope of the disclosure. It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present. When referring to a “back” or “front” side or end of an imaging component, it will be understood that front refers generally to the side from which imaging would be directed and back as generally the opposite side.

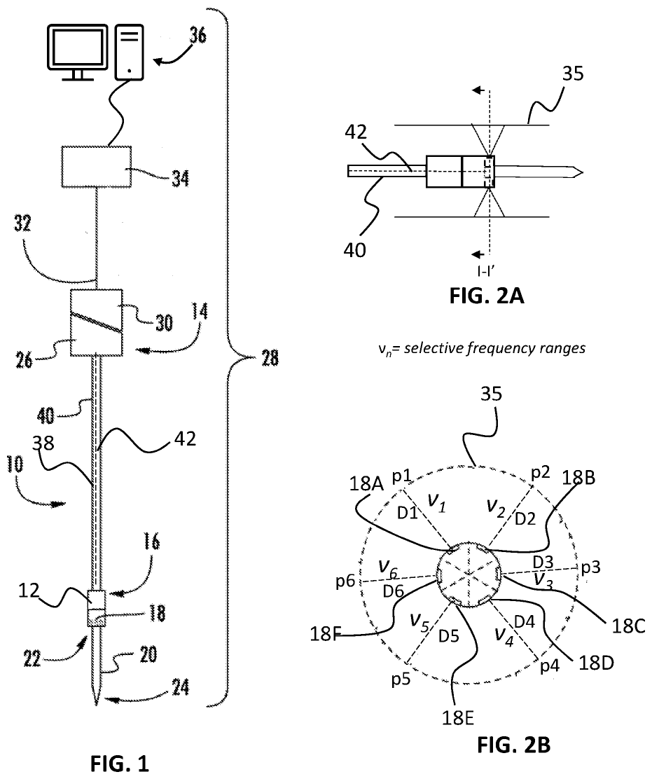

FIG. 1 is an illustrative diagram of an ultrasound catheter probe system 28 according to some embodiments. An ultrasound imaging probe 10 includes a body member 40 having a proximal end 14 and a distal end 16. The probe 10 includes a plurality of transducers 18. Probe 10 also includes an elongated tip 20 having a proximal end 22 and a distal end 24. Probe 10 includes a proximal connector 26 which connects probe 10 to other components of system 28, including a computer system 36. In an embodiment of the invention, the medical device 10 is part of a system 28 that includes a distal connector 30, electrical conductor 32, a data acquisition unit 34 and a computer system 36.

In some embodiments, body member 40 is tubular and has a central lumen for containing various connectors and channels (e.g., conductor 42) that extend toward distal end 16. In some embodiments, body member 12 has a diameter of about 650 μm or less. These dimensions are illustrative and not intended to be limiting. In some embodiments, the diameter of the probe 10 will depend on the type of device that probe 10 is integrated with and where the probe 10 will be used (e.g., in a blood vessel), which will become apparent to those of ordinary skill in the art in view of the present disclosure.

The proximal end 14 of the body member 12 is attached to the proximal connector 26. In some embodiments, probe 10 includes an elongated tip 20 in which its proximal end 22 is attached to the distal end 16 of body member 12. The elongated tip 20 may be constructed with an appropriate size, strength, and flexibility to be used for guiding probe 10 through a body lumen (e.g., a blood vessel). Elongated tip 20 and/or other components of probe 10 may include a radio-marker (e.g., visible to angiography) for precisely guiding the catheter through a lumen and positioning transducers 18 in the desired location. In some embodiments, probe 10 and distal end 16 are constructed and arranged for rapid exchange use. Body member 12 and elongated tip 20 may be made of resilient flexible biocompatible material such as is common for IVUS and intravascular catheters known to those of ordinary skill in the art.

Probe 10 has a tubular body with a central lumen 38. In some embodiments, probe 10 may have lumens for use with various features not shown (guidewires, fiberoptics, saline flush lumens, electrical connectors, etc.). In some embodiments, the outer diameter of the body member 12 and elongated tip 20, if present, is substantially consistent along its length and does not exceed a predetermined amount.

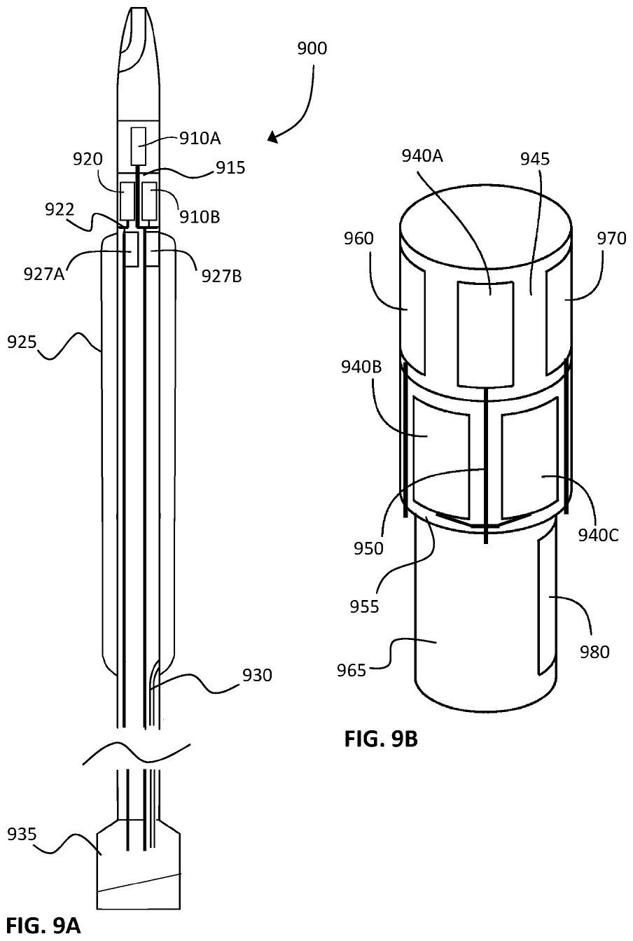

At least two transducers 18 share a shared conductor 42 to which they are each connected. Shared conductor 42 carries electromagnetic signals generated by the attached transducers to a data acquisition unit 34 (e.g., including an analog-to-digital converter). Signals received and processed by data acquisition unit 34 are then processed by a computer system 36 programmed to store and analyze the signals (e.g., calculate distance measurements between the catheter and lumen wall). In some embodiments, by sharing conductors (e.g., conductor 42) the space saved within body member 12 may be utilized to incorporate additional features (e.g., an expandable balloon and a balloon media lumen such as shown in FIG. 9A. In some embodiments, the diameter of body member 40 may be reduced as the number of shared conductors is increased, the overall number of conductors reduced, while the number of transducers is not decreased.

In some embodiments, ultrasound transducers 18 are piezoelectric. The transducers may be built using piezoelectric ceramic or crystal material and layered by one or more matching layers that can be thin layers of epoxy composites or polymers. In some embodiments, the transducers are PMUTs (Piezoelectric Micromachined Ultrasonic Transducers), CMUTs (Capacitive Micromachined Ultrasonic Transducers), and/or photoacoustic transducers.

The operating frequency for the ultrasound transducers may be in the range of from about 8 to about 50 MHz or even up to about 60 MHz, depending on the dimensions and characteristics of the transducer and requirements of the particular application. Generally, higher frequency of operation provides better resolution and a smaller medical device 10. However, the tradeoff for this higher resolution and smaller catheter size may be a reduced depth of penetration into the tissue of interest and increased echoes from the blood itself (making the image more difficult to interpret). Lower frequency of operation is more suitable for imaging in larger vessels or within structures such as the chambers of the heart. Although specific frequency ranges have been given, these ranges given are illustrative and not limiting. The ultrasonic transducers 18 may produce and receive any frequency that leaves a transducer 18, impinges on some structure or material of interest and is reflected back to and picked up by a transducer 18. The center resonant frequency and bandwidth of a transducer is generally related to the thickness of transducer materials generating or responding to ultrasound signals. For example, in some embodiments, a transducer includes a piezoelectric material such as quartz and/or lead-zirconate-titanate (PZT). A thicker layer will generally respond to a longer wavelength and lower frequency and vice versa. For example, a 50 micron thick layer of PZT will have a resonant frequency of about 40 MHz, a 65 micron thick layer will have a resonant frequency of about 30 MHz, and a 100 micron layer will have a resonant frequency of about 20 MHz. As further described herein, matching and backing layers may be included which affect the bandwidth and other characteristics of a transducer.

In some embodiments, particular transducers 18 that are connected to a shared conductor are adapted to generate responsive signals to distinct frequency ranges of incident ultrasound. In some embodiments, the frequency range of a first transducer sharing a conductor is between about 8 and 30 MHz and the frequency range of a second transducer is between about 30 and 50 MHz. For example, the first transducer may be configured with a PZT layer of between about 60 and 100 microns and have a resonant frequency of between about 20 and 30 MHz and the thickness of a PZT layer of the second ultrasound transducer can be between about 40 and 60 microns and have a resonant frequency of between about 30 and 50 MHz. In some embodiments, a frequency range for a first transducer may be configured between 20 and 35 MHz and a frequency range of a second transducer may be configured between 35 and 50 MHz. In some embodiments, the transducers use piezoelectric crystals composed of Pb(Mg_{1/3}Nb_{2/3})O₃—PbTiO₃ (PMN-PT) or other types of piezoelectric materials with dimensions configured to resonate, for example, at the disclosed frequencies.

In some embodiments, the respective ranges are separated by about 10 MHZ or more. In some embodiments, a resonant frequency of one transducer may be centered around 20, 25, or 30 MHz while another transducer sharing a conductor may have a resonant frequency centered around 35, 40, 45, or 50 MHz, for example. The respective materials and dimensions of the transducer layers may be configured accordingly. As further described herein, the system may be programmed to cause the particular transducers to selectively transmit the separate frequency ranges, receive and forward corresponding echo signals to the computer system, and subsequently correlate the echo signals to the signals transmitted by the respective transducer.

In some embodiments, probe 10 is connected with an actuating mechanism that may rotate and/or longitudinally move at least some portions of probe 10 and its transducers 18. A controlled longitudinal and/or radial movement permits the probe to obtain ultrasound readings from different perspectives within a surrounding structure, for example. Positioning the probe and its transducers in target locations may be augmented/guided by real-time imaging feedback provided by the transducers and system 28. Relative positions of the probe may be tracked and recorded during such processes (e.g., by using an encoder or other position sensing tool).

In some embodiments, system 28 is programmed to analyze and identify characteristics of the medium (e.g., blood) between probe 10 and structure in order to determine where the medium ends with respect to the structure (e.g., blood vessel wall). In some embodiments, multiple ultrasound images of the blood may be generated and the differences between the images are used to identify movement/change of the blood over time (e.g., as a result of a heart pumping). In some embodiments, doppler echo signals are used to determine these differences. Because the blood vessel wall does not have the same movement/change characteristics as the blood, the amount (or distance) between the probe 10 and blood vessel wall can be calculated. In some cases, reliance on the blood images without substantial reliance on images of the blood vessel wall may be used to determine the distance between probe 10 and blood vessel wall.

FIG. 2A is an illustrative side perspective diagram of an ultrasound catheter probe placed within a lumen according to some embodiments. FIG. 2B is a cross-sectional perspective diagram of the ultrasound catheter probe across lines I-I′ of FIG. 2A. Catheter probe 10 is shown inserted into a lumen 35. Two transducers 18A and 18B of transducers 18 share conductor 42. Connected computer system 36 is programmed to cause transducer 18A to generate a first pulse within a first frequency range v₁ and cause transducer 18B to generate a second pulse within a second frequency range v₂ over a time interval where each of the pulses is incident on different portions of lumen 35. In response to echoes from lumen 35, transducers 18A and 18B generate electromagnetic signals respective to the first and second pulses that reflect the first and second frequency ranges v₁ and v₂. These electromagnetic signals are both transmitted through shared conductor 42 to a signal processor and computer system 36.

Computer system 36 is programmed to analyze and distinguish between the echoes associated with respective pulses. This may be performed by identifying the characteristics of the signals associated with the first and second frequency ranges v₁ and v₂ of the first and second pulses. Other pulses may be similarly delivered/echoed using other transducers 18 at frequency ranged v₃, v₄, v₅, and v₆. In some embodiments, these pulses may be delivered simultaneously or at different times. For example, frequency ranges v₃ and v₄ may be delivered at the same time and same frequency range apart from frequency ranges v₂ and v₆, which may be delivered at a separate time. Along with identifying and associating the signals with respective transducers, the computer system 36 is programmed to analyze the signals and calculate a radial distance measurement (e.g. D1, D2, . . . , D6) between each transducer 18 and lumen 35. This may be done, for example, by utilizing time-of-flight information of the echo signals and previously determined/differentiated signatures representative of a lumen wall (e.g., of lumen 35) and a particular medium (e.g., blood) between the transducer and lumen 35. Exemplary systems and methods for making such calculations are described, for example, in U.S. Pat. No. 10,231,701 filed Mar. 14, 2014 (the '701 Patent), the entire contents of which is herein incorporated by reference.

Based on distance calculations (D1, D2, . . . , D6), the shape and dimensions of lumen 35 may be estimated by further utilizing information including the dimensions of probe 10 and applying interpolation and/or other mathematical fitting techniques. For example, the relative positions of points (p1, . . . , p6) about lumen 35 may first be calculated and a curve fitting algorithm (e.g., spline interpolation) is applied to generate a two-dimensional slice representation of the lumen 35. As described in the '701 Patent, multiple slices can be calculated by taking sets ultrasound readings along the longitudinal extent of lumen 35 and combining them to generate a three-dimensional representation.

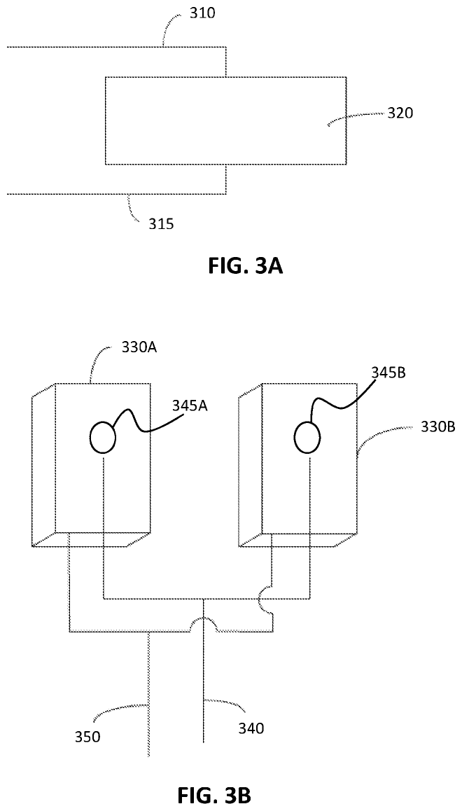

FIG. 3A is an illustrative diagram of a transducer with a dedicated conductor. In a traditional ultrasound system, a transducer 320 includes a conductor with a signal conductor 315 and a ground conductor 310 in which the signal conductor is not shared with additional transducers. Signal conductors for respective transducers in a traditional system remain isolated in order to avoid cross-signaling and interference between transducer signals.

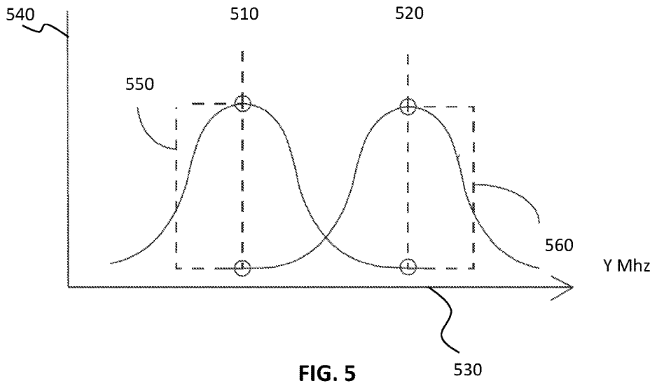

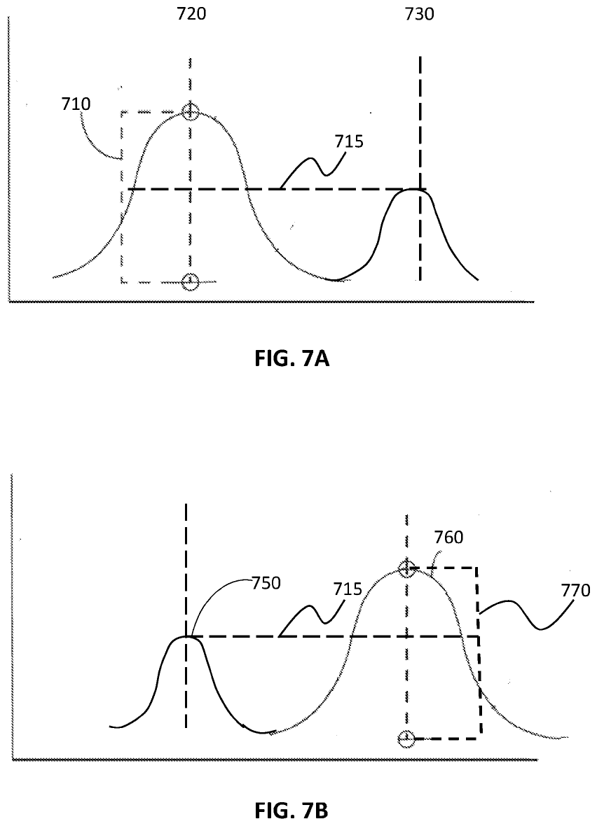

FIG. 3B is an illustrative diagram of multiple transducers sharing a conductor according to some embodiments. A signal conductor 340 is shared among and connected to both transducers 330A and 330B at electrical connectors 345A and 345B, respectively. A ground conductor 350 is also shared and connected to both transducers 330A and 330B. Each transducer 330A and 330B is constructed to be selectively responsive to particular ultrasonic frequency ranges such as, for example, shown and described in reference to FIGS. 5A, 7A and 7B. When each transducer is activated by incident ultrasound signals corresponding to their respective selective frequency ranges, they each responsively produce corresponding electromagnetic signals that are transmitted through electrical connectors 345A and 345B and through shared conductor 340.

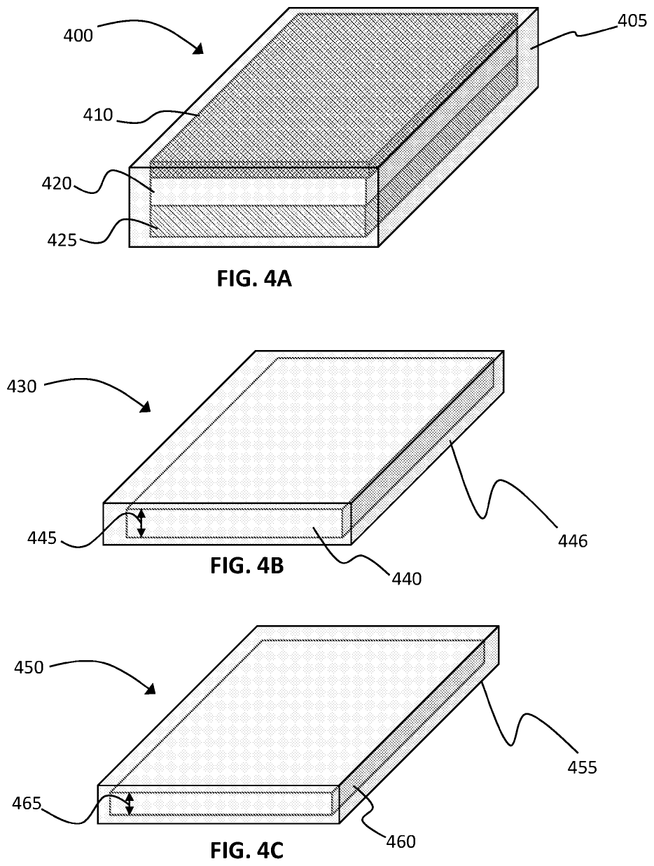

FIG. 4A is an illustrative diagram of a piezoelectric transducer. A piezoelectric transducer 400 includes a piezoelectric crystal 420, a backing layer 425, a matching layer 410, and a protective cover 405. The piezoelectric crystal is constructed to mechanically vibrate in response to ultrasonic waves incident upon the transducer and, in response, generate a voltage across the crystal. This charge differential may be carried through a connected conductor (e.g., similar to the conductors shown in FIGS. 1, 2A, 3A, and 3B). The variance in charge across the crystal may be correlated with ultrasonic frequencies incident upon the crystal over time (e.g., by a computer system 36 and as represented in the chart of FIG. 5A). Similarly, an electrical charge may be introduced across the crystal via connected conductors and an external electric power source and cause the crystal to emit ultrasonic waves. Such an emission may be used to deliver ultrasound to external structures, after which a responsive signal (e.g., echo signals) may be monitored to detect the presence and characteristics of those structures.

Backing layer 425 may be configured and arranged to reduce excess reverberation (i.e., noise) in the transducer caused by excitation of crystal 420. Matching layer 410 may be constructed of polymers or other materials with particular ultrasonic characteristics and is utilized to expand (“ring-down”) the range of frequencies to which the transducer emits and responds (e.g., for enhancing distinguishing characteristics of imaged tissues). A protective layer 405 may be used to envelope the transducer and insulate its components from environmental factors and damage.

FIG. 4B is an illustrative diagram of a transducer configured for a selective response to a frequency range according to some embodiments. Transducer 430 includes a piezoelectric layer 440 constructed with a particular thickness 445 and other dimensions to resonate at a relatively narrow frequency range and, in some embodiments, includes minimal or no matching layer compared to traditional imaging transducers. In some embodiments, for example, transducer 430 may be constructed using a piezoelectric layer 440 with a thickness 445 of about 40 to 100 microns for a resonance frequency between about 20 and 50 MHz.

In some embodiments, a transducer includes a matching layer having a thickness of less than about a quarter of the ultrasound wavelength for the targeted frequency range and, in some embodiments, no further matching layer. In some embodiments, a conductive material (e.g., a polymer or adhesive) may be configured and utilized as both a connecting electrode for the transducer and as a “matching layer.” The materials may be composed to gradually transition the impedance of ultrasound waves between the piezoelectric layer 440 and imaging targets. In some embodiments, the electrode is applied as a common layer over multiple transducers (e.g., transducers 18) after they are inserted into an imaging probe (e.g., probe 10 of FIG. 1).

In some embodiments, a backing layer is omitted or substantially omitted and permits the transducer to occupy a smaller footprint such as within an intravascular (e.g., coronary) probe. Noise that is associated with a reduced or omitted backing layer may be accounted for (e.g., utilizing software or hardware) to an extent needed to distinguish between the presence of a structure barrier (e.g., a lumen wall) and an intermediate medium (e.g., blood). In some embodiments, protective layer 446 is omitted and the transducer may be sufficiently isolated by other components (e.g., a biocompatible sealing layer/membrane placed over a probe after transducers are inserted into a probe).

FIG. 4C is an illustrative diagram of a transducer configured for selectively responding to another frequency range according to some embodiments. A piezoelectric crystal 460 is constructed with a thickness 465 and protected with a protective layer 455. In some embodiments, thickness 465 is different from the thickness 445 of transducer 430 so that transducers 430 and 450 selectively respond to different ranges of frequencies. For example, transducer 450 may resonate at a frequency range of between about 20 and 30 MHz while transducer 430 resonates at a frequency range of between about 30 and 50 MHz. In some embodiments, the frequency ranges are separated by at least about 10 MHz. Transducers 430 and 450 may be integrated into an imaging probe (e.g., probe 10 of FIGS. 1 and 2A) and share a conductor as described further herein. This way, when a system (e.g., system 28) obtains a signal through the shared conductor, the system can distinguish between a signal from transducer 430 and 450.

FIG. 5 is an illustrative chart of exemplary frequency responses of transducers sharing a conductor according to some embodiments. A horizontal axis 530 represents frequency (Mhz) while a vertical axis 540 represents signal amplitude (dBs). A first transducer is constructed to have a resonant frequency centered at 510. A second transducer is constructed to have a resonant frequency centered at 520. In some embodiments, the frequencies at 510 and 520 are separated by a sufficient amount in frequency (e.g., 10 MHz) and/or amplitude so that incident ultrasound signals within approximated variances of the respective resonant frequencies cause uniquely identifiable electromagnetic signals to be produced by the respective transducers. Thus, when corresponding electromagnetic signals are produced, a system (e.g., system 28) can be programmed to identify which of the transducers produced the signals.

In some embodiments, signals from one transducer (e.g., corresponding to resonant frequency 510) responsive to a resonance frequency are segregated within a particular frequency and amplitude differential 550 while a different transducer sharing the same conductor (e.g., corresponding to resonant frequency 520) is more particularly responsive to a particular frequency and amplitude differential 560 corresponding to a different resonance frequency.

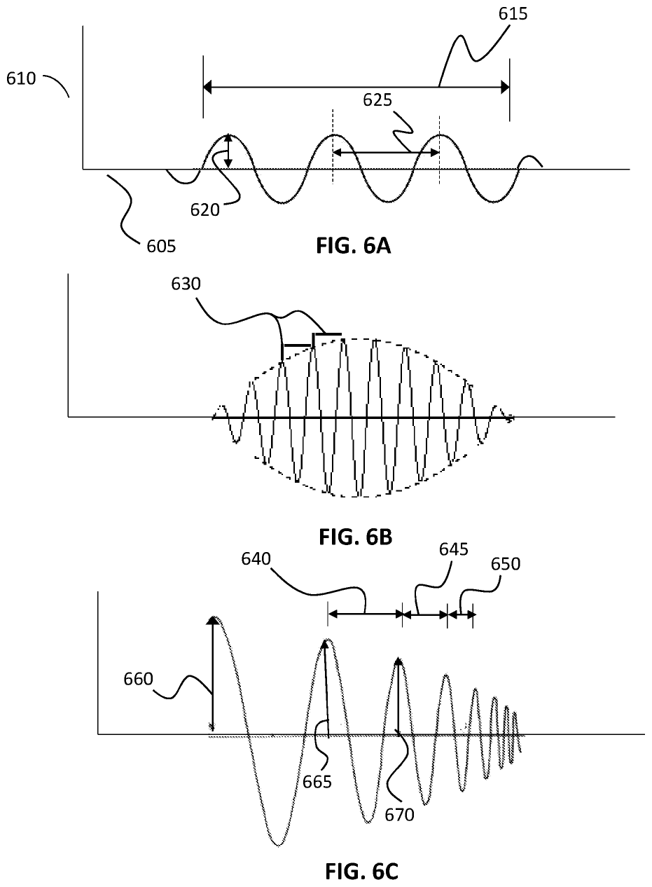

FIG. 6A is an illustrative chart of a modulated electric pulse for activating a transducer according to some embodiments. A horizontal axis 605 represents time while a vertical axis 610 represents voltage. In order to generate an ultrasound pulse (or “chirp”) from a piezoelectric transducer via an electric conductor, a voltage can be generated through the conductor and across the transducer's ultrasound-generating component (e.g., piezoelectric layer). These currents can be configured to control the transmitted ultrasound pulse including the frequency, intensity, and length.

In some embodiments, a modulated pulse such as shown in FIG. 6A includes a continuous sinusoidal voltage having a particular period 625 with a maximum amplitude 620 over a particular time interval 615. In some embodiments, the time interval, frequency, and intensity of the voltage cause a transducer of particular dimensions, structure, and material to substantially resonate at or about its particular resonance frequency. In some embodiments, the voltage is modulated at a frequency at or about the same frequency as a targeted transducer to maximize resonance intensity.

FIG. 6B is an illustrative chart of an intensity-envelope of a modulated electric pulse which may be used in some embodiments to activate a transducer according to some embodiments. In some embodiments, a pulse time interval of about 100 nanoseconds or more and a frequency of between 20 and 30 MHz are used to activate a piezoelectric transducer with a resonance frequency of between about 20 and 30 MHz and that does substantially activate a transducer with a resonance frequency of between about 40 and 50 MHz. In some embodiments, a voltage of about 150 volts or less is used at the height of the envelope. The voltage of an envelope is shown stepping upward at 630. To activate another transducer sharing a conductor with a resonance frequency of between about 40 and 50 MHz, a pulse time interval of for about 100 nanoseconds at a frequency of between about 40 and 50 MHz is used.

FIG. 6C is an illustrative chart of a frequency-and intensity-modulated electric pulse for activating a transducer according to some embodiments. In some embodiments, the frequency and amplitude of applied voltage is varied over time during a pulse. The peak amplitudes of a sinusoidal pulse decreases between 660, 665, and 670 while the frequency increases between 640, 645, and 650. In some embodiments, the frequency during the pulse is closest to that of the target transducer resonance frequency when the amplitude of voltage is at or near its maximum while the frequency during the pulse is closest to that of another transducer (not to be substantially activated) sharing a conductor when the amplitude of voltage is at or near its minimum.

FIGS. 7A and 7B are illustrative charts of a frequency response from multiple transducers sharing a conductor. As described herein, the resonant frequency for a transducer can be activated via a shared conductor without substantially activating the resonant frequency of other transducers sharing the same conductor. FIG. 7A illustrates the resonant frequency at 720 of a first transducer activated at an amplitude at or above about level 715 while the resonant frequency at 730 of a second transducer that is not (substantially) activated is not at or above an amplitude at about level 715. In some embodiments, a response differential at 710 is used to analyze and differentiate responsive signals received at the first transducer. FIG. 7B illustrates the resonant frequency at 760 of the second transducer activated at or above an intensity at about position 715 while the resonant frequency at 750 of the first transducer is not (substantially) activated at or above an intensity at about position 715. In some embodiments, a response differential at 770 is used to analyze and differentiate responsive signals received at the first transducer.

In some embodiments a lower frequency signal may cause both of the transducers sharing the same conductor to activate despite only one transducer being designed to resonate with the signal frequency. In these instances, a computer system 36 may be designed to identify the response from the transducer with a higher resonate frequency which was still substantially activated by the lower frequency signal. Once the response signal from the higher resonate frequency transducer is identified a computer system 36 can be programmed to isolate the response signal from the transducer with a lower resonate frequency reducing any substantial interference.

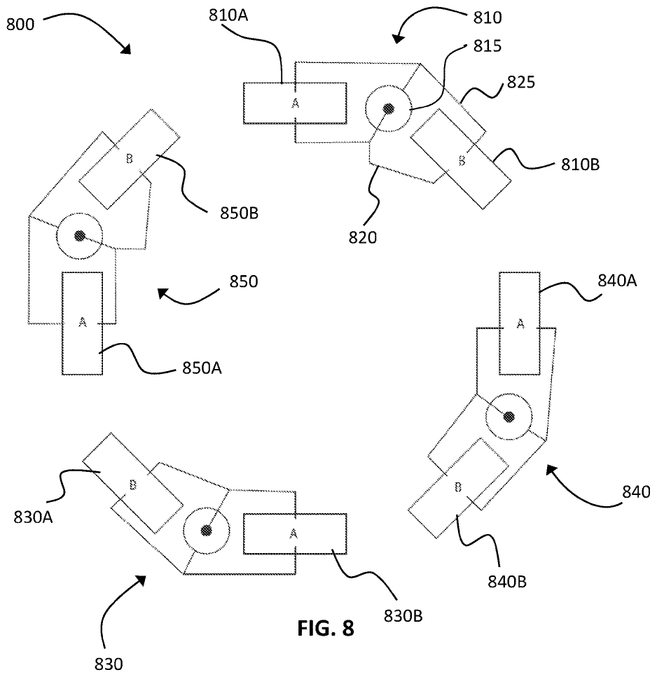

FIG. 8 is an illustrative diagram of multiple pairs of transducers in which each pair shares a conductor, in accordance with some embodiments. Four pairs of transducers, 810A and 810B, 840A and 840B, 830A and 830B, and 850A and 850B are arranged in a circular array 800 (e.g., around body 12 of FIG. 1), each of the pairs sharing a conductor. Pair 810A and 810B share a signal conductor 820 and ground conductor 825 that are fed through a contact area 815. Individual transducers of each pair can be configured to be responsive to different ranges of wavelengths such as described, for example, with reference to FIGS. 4 and 5.

In some embodiments, a connected system (e.g., system 28 of FIG. 1) is programmed to selectively activate a first transducer of a pair by delivering electromagnetic signals corresponding to the first transducer's frequency range through a shared conductor. This way, the second transducer of a pair is not activated by signals until a different electromagnetic signal is delivered to the shared conductor that corresponds to the second transducer's frequency range.

In some embodiments, electromagnetic signals are delivered selectively to activate alternate transducers of the circular array 800. For example, during one time interval, a first set of transducers 810A, 850A, 830A, and 840A are activated while the remaining transducers are not activated. In some embodiments (e.g., from within a blood vessel), the transducers are configured so that echo readings returned in response to the delivered signals will be within the frequency differential corresponding to the respective source transducers. After readings are collected for the first set of transducers, a second set including the remaining transducers may be activated using their particular frequency ranges. The results of the readings may then be combined and used to calculate attributes of surrounding structures and mediums such as further described herein.

Sharing transducers such as shown in FIG. 8 allows for the use of fewer conductors with a relatively greater number of transducers, providing benefits such as enhanced data collection and scope with a narrower catheter footprint. Other variations and combinations of shared conductors and activation sequences may be employed depending on the application. For example, opposing transducers (e.g., transducers 810A and 830A) may be activated during one sequence and transducers 840A and 850A used as “side channels” to collect complimentary signals generated in response to activating transducers 810A and 830A. These side channels may be used to make additional distance measurements between a catheter and imaged structure and/or used to refine/confirm data/measurements collected/calculated through transducers from which the source signals originated.

FIG. 9A is an illustrative diagram of an ultrasound catheter having shared conductors according to some embodiments. A catheter 900 includes transducers 910A and 910B that share a conductor 915 such as described in various embodiments herein. Transducers 910A and 910B are located at different positions along the longitudinal axis of catheter 900. A transducer 920 is located at the same longitudinal position as transducer 910B but utilizes a conductor 922 disconnected from conductor 915 and transducers 910A and 910B. In some embodiments, a system (e.g., system 28) is programmed to activate transducers 910A and 920 simultaneously to obtain readings from two separate longitudinal and radial positions (e.g., of a body lumen) along catheter 900. A signal is delivered to selectively activate transducer 910A while not activating transducer 910B through their shared conductor. During another time interval, transducer 910B and/or other transducers may be activated in order to obtain readings around their respective locations.

Catheter 900 includes an expandable balloon 925 (e.g., an angioplasty balloon) which can be expanded or deflated by controlling the introduction or expulsion of a medium (e.g., air or saline) through a lumen 930. In some embodiments, readings from transducers 910A, 910B, and 920 are utilized to position balloon 925 in an optimal location for deploying the balloon 925 (e.g., within a diseased body vessel). Balloon 925 may also be utilized and expanded to center or hold catheter 900 in a particular position within a structure. Catheter 900 also includes a connector 935 for connecting catheter 900 with catheter system components (e.g., a computer, signal processor, balloon media source). In some embodiments, transducers 927A and 927B are located within balloon 925. These transducers may be used, for example, to monitor the level of expansion of balloon 925. Balloon 925 may be made of a material or include a coating that enhances their ultrasound reflectivity.

FIG. 9B is an illustrative diagram of a transducer arrangement with shared conductors according to some embodiments. A catheter body segment 945 along a particular longitudinal span includes transducers 960, 970, and 940A which are rotationally staggered with respect to each other. Another catheter body segment 955 includes transducers 940B and 940C wherein transducers 940A, 940B, and 940C share a conductor 950 that is not connected to transducers 960 and 970. In some embodiments, transducers 940A and 940C are activated to take readings during the same time interval while transducer 940B is not activated. This may be accomplished, for example, by configuring transducers 940A, 940B, and 940C with different frequency response profiles and delivering signals to them correspondingly. The signals returned in response to the activations can be separated from each other such as through hardware and/or software filtering.

Another catheter body segment 965 along a different longitudinal span includes a transducer 980. Segment 965 is configured so that transducer 980 is positioned at a different radial distance from other transducers relative to the center of catheter 900. The different radial distance permits transducer 980 to take images in coordination with other transducers located on different segments (e.g., segments 955 and 945) at differing radial distances, which may provide complementary imaging information with respect to surrounding structure. Other transducers may be positioned on segment 965 and may share conductors with transducer 980 or those attached to other segments in accordance with some embodiments herein.

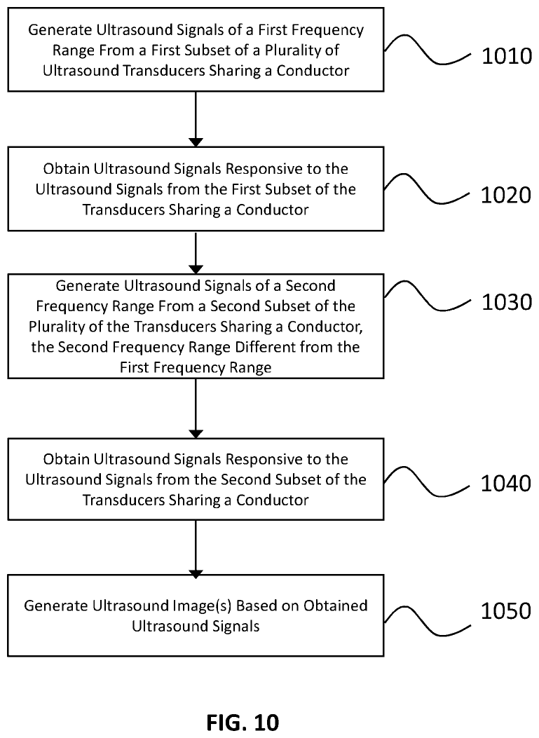

FIG. 10 is a block diagram of a process for generating an ultrasound image using transducers sharing a conductor according to some embodiments. At block 1010, ultrasound signals of a first frequency range corresponding to a first transducer of a plurality of transducers on a probe (e.g., probe 10 of FIG. 1) are generated using a shared conductor connected to the first transducer. The ultrasound signals may be generated using an electric power source (e.g., as part of system 28 and controlled by computer system 36) and transmitting a pulse according to FIGS. 6A, 6B, and 6C through the shared transducer. The ultrasound signals are transmitted toward a structure (e.g., a blood vessel wall) proximate to the transducer that may be separated from the transducer by a medium (e.g., blood). At block 1020, ultrasound signals responsive to the transmitted signals of block 1010 are obtained. The signals echoed and obtained in response to the first transducer will activate the first transducer, from which representative electric signals will be transmitted back through the shared conductor to a signal processor (e.g., as shown in FIG. 1). In some embodiments, the responsive signals are also obtained by other transducers other than the first transducer (e.g., side channels). In some embodiments, additional transducers of the plurality of transducers are activated at the same time as the first transducer.

At block 1030, ultrasound signals of a second frequency range corresponding to a second transducer of the plurality of transducers on the probe are transmitted through the shared conductor. In some embodiments, the first and second frequency ranges do not overlap and the respective corresponding transducers are not substantially activated by the other of the respective frequency ranges (e.g., as shown in FIGS. 7A and 7B). At block 1040, ultrasound signals responsive to the signals transmitted in the second frequency range are obtained. These signals may represent features of structure and media at a relatively different position with respect to the probe that were obtained at block 1020. For example, these signals may represent different radial and/or longitudinal positions about the probe such as shown in FIGS. 1, 2, 8 and 9.

At block 1050, based on the signals received at blocks 1020 and 1040 one or more ultrasound images are generated. The signals received may represent echoes of media and/or structure at different positions about the probe. In some embodiments, signals received from the transducers are combined to generate an image representing a full 360 degree perspective around the probe or a longitudinal extent of structure (e.g., a blood vessel) along the probe. In some embodiments, intensity values or other characteristics of the image(s) can be used to calculate distances from the probe such as further described herein. Based on these distances and known dimensions of the probe, the diameters of a surrounding structure may be calculated through different radial axis calculated between the probe and the structure, from which a shape and size of a cross section of structure may be further determined such as described in the '701 Patent. In some embodiments, these cross-sectional calculations are made at different longitudinal positions of the probe, providing a three-dimensional perspective along a longitudinal extent of the structure.

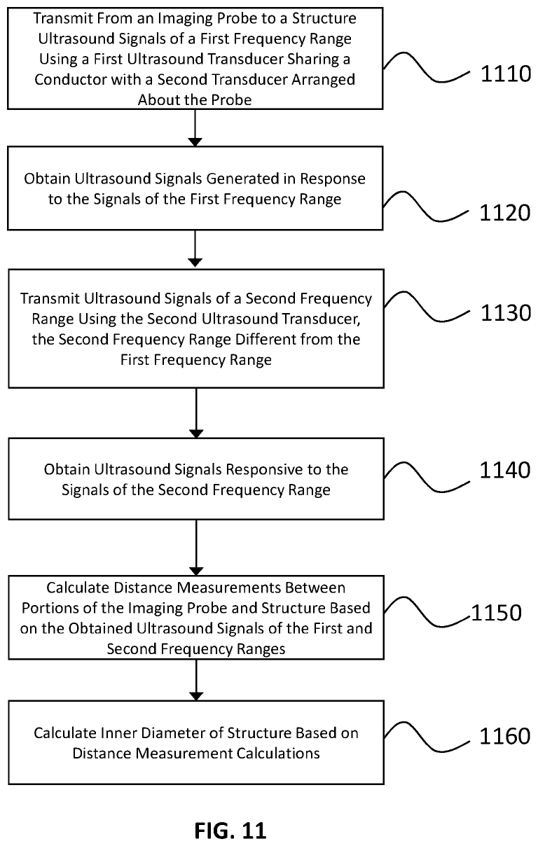

FIG. 11 is a block diagram of a process for using an imaging probe with transducers sharing a conductor to calculate distance measurements according to some embodiments. A first transducer and a second transducer are arranged on the probe and share a conductor (e.g., as shown in FIGS. 1, 2, 3, 8, and 9). At block 1110, ultrasound signals are transmitted from a first transducer within a first frequency range (e.g., as described in reference to FIGS. 6 and 7). In some embodiments, the second transducer does not substantially generate signals or signals above a predetermined intensity compared to signals from the first transducer (e.g., as shown in FIGS. 7A and 7B). The signals may be transmitted toward a structure, for example, a blood vessel surrounding the probe inserted into the vessel.

At block 1120, ultrasound signals responsive to those transmitted at block 1110 are obtained. These signals may be ultrasound echo signals representing features of the structure (e.g., a blood vessel) and/or a medium (e.g., blood) between the probe and structure. The signals echoed and obtained in response to the first transducer will activate the first transducer, from which representative electric signals will be transmitted back through the shared conductor to a signal processor (e.g., as shown in FIG. 1). In some embodiments, signals from the second transducer, if any, will not substantially impact signals transmitted and obtained by the first transducer. For example, as described further herein, they may be configured to be that of a substantially lower intensity and/or of a different frequency that may be filtered out (e.g., frequency matching) either through electrical components or software signal processing.

At block 1130, ultrasound signals are generated and transmitted by the second transducer within a second frequency range different from the first frequency range (e.g., as described in reference to FIGS. 6 and 7). The ultrasound signals transmitted by the second transducer may be directed toward different areas of the structure and/or intervening medium than that by the first transducer (e.g., as shown and described in reference to FIGS. 2 and 8). At block 1140, signals generated in response to the second frequency range are obtained. These signals may be obtained by the second transducer tuned to the frequencies it generated.

At block 1150, the signals obtained at blocks 1120 and 1140 are analyzed and used to calculate distances between the imaging probe and the structure(s) toward which the ultrasound signals were transmitted at blocks 1110 and 1130. For example, echo signals may be used to calculate a distance from the transducer to the structure that the echo signals reflect (e.g., as described in the '701 Application). At block 1160, the inner diameter of the structure is calculated based on the distance calculations of block 1150. As described in the '701 Application, for example, the inner diameter may be calculated by using known dimensions of the imaging probe and relative positions of the transducers with respect to the imaging probe. Multiple inner diameters calculated based on multiple distance calculations from circularly arranged transducers may be used to generate cross-sectional or 3-dimensional dimensions and/or shapes of the structure.

The processes described herein (e.g., the processes of FIGS. 10 and 11) are not limited to use with the hardware shown and described herein. They may find applicability in any computing or processing environment and with any type of machine or set of machines that is capable of running a computer program. The processes described herein may be implemented in hardware, software, or a combination of the two. The processes described herein may be implemented in computer programs executed on programmable computers/machines that each includes a processor, a non-transitory machine-readable medium or other article of manufacture that is readable by the processor (including volatile and non-volatile memory and/or storage elements), at least one input device, and one or more output devices. Program code may be applied to data entered using an input device to perform any of the processes described herein and to generate output information.

The processing blocks (for example, in the processes of FIGS. 10 and 11) associated with implementing the system may be performed by one or more programmable processors executing one or more computer programs to perform the functions of the system. All or part of the system may be implemented as, special purpose logic circuitry (e.g., an FPGA (field-programmable gate array) and/or an ASIC (application-specific integrated circuit)). All or part of the system may be implemented using electronic hardware circuitry that include electronic devices such as, for example, at least one of a processor, a memory, a programmable logic device, and/or a logic gate.

The processes described herein are not limited to the specific examples described. For example, the process of FIGS. 10 and 11 are not limited to the specific processing orders illustrated. Rather, any of the processing blocks of FIGS. 10 and 11 may be re-ordered, combined or removed, performed in parallel or in serial, as necessary, to achieve the results set forth above.

Elements of different embodiments described herein may be combined to form other embodiments not specifically set forth above. Various elements, which are described in the context of a single embodiment, may also be provided separately or in any suitable subcombination. Other embodiments not specifically described herein are also within the scope of the following claims.

1. An ultrasound probe, the probe comprising:
a flexible body elongated along a longitudinal axis and assembled for insertion into a structure;
a plurality of ultrasound transducers arranged along the flexible body;
a shared signal conductor shared among a first and second of the plurality of ultrasound transducers, the first transducer configured to respond to a first ultrasound frequency range and the second transducer configured to respond to a second ultrasound frequency range different from the first ultrasound frequency range.

2. The ultrasound probe of claim 1, wherein the first frequency range is between about 20 and 35 MHz and the second frequency range is between about 35 and 50 MHz.

3. The ultrasound probe of claim 2, wherein a resonant frequency difference between the first and second transducer is at least about 10 MHz.

4. The ultrasound probe of claim 1, wherein the first transducer comprises a first piezoelectric layer having dimensions configured to cause the first layer to resonate within the first ultrasound frequency range and the second transducer comprises a second piezoelectric layer having dimensions configured to cause the second layer to resonate within the second ultrasound frequency range.

5. The ultrasound probe of claim 1, wherein a piezoelectric layer of the first ultrasound transducer has a thickness of between about 60 and 100 microns and a resonant frequency of between about 20 and 35 MHz and wherein a piezoelectric layer of the second transducer has a thickness between about 40 and 60 microns and a resonant frequency of between about 35 and 50 MHz.

6. The ultrasound probe of claim 4, wherein neither of the first and second transducers include a conductive matching layer.

7. The ultrasound probe of claim 4 wherein a conductive electrode is layered over the first and second transducers.

8. The ultrasound probe of claim 4, wherein at least one of the first and second transducers does not include a matching layer of a thickness that is equal to or greater than about a quarter ultrasound wavelength corresponding to the respective first and second frequency ranges.

9. The ultrasound probe of claim 1, wherein the plurality of ultrasound transducers are arranged circumferentially about the flexible body.

10. The ultrasound probe of claim 2, wherein the first ultrasound transducer and second ultrasound transducer are circumferentially adjacent to each other among the plurality of ultrasound transducers.

11. The ultrasound probe of claim 3, comprising third and fourth ultrasound transducers of the plurality of transducers that share a conductor, the third and fourth ultrasound transducers arranged circumferentially across from the first and second ultrasound transducers, respectively.

12. The ultrasound probe of claim 1, further comprising a therapeutic device arranged at a predetermined location with respect to the plurality of transducers.

13. The ultrasound probe of claim 12 wherein the therapeutic device comprises an angioplasty balloon.

14. The ultrasound probe of claim 13, wherein one or more of the plurality of transducers are arranged within the angioplasty balloon.

15. An ultrasound system for measuring the dimensions of a structure, the system comprising:
a flexible body elongated along a longitudinal axis and assembled for insertion into the structure;
a plurality of ultrasound transducers arranged circumferentially about the flexible body;
a shared signal conductor shared among a first and a second ultrasound transducer of the plurality of ultrasound transducers, the shared conductor arranged to transmit electrical signals from the first and second ultrasound transducers, the first transducer configured to respond to a first ultrasound frequency range and the second transducer configured to respond to a second ultrasound frequency range different from the first ultrasound frequency range;
one or more processors programmed and configured to calculate a plurality of distances between the elongate flexible body and an inner wall of the structure, the calculating based on the electrical signals transmitted from the plurality of ultrasound transducers.

16. The ultrasound system of claim 15, wherein the one or more processors are further configured to calculate cross-sectional dimensions and shapes of the structure based on the plurality of distances.

17. The ultrasound system of claim 15, wherein the first frequency range is between about 20 and 30 MHz and the second frequency range is between about 30 and 50 MHz.

18. The ultrasound system of claim 15, wherein the one or more processors are programmed and configured to:
transmit a first set of signals through the shared conductor to cause the first transducer to transmit ultrasound signals of the first frequency range toward the structure;
obtain and process ultrasound signals responsive to the first set of signals;
after obtaining the ultrasound signals responsive to the first set of signals, transmit a second set of signals through the shared conductor to cause the second transducer to transmit ultrasound signals of the second frequency range toward the structure; and
obtain and process ultrasound signals responsive to the second set of signals;
wherein the calculating a plurality of distances between the elongate flexible body and an inner wall of the structure is based on analyzing the processed signals responsive to the first and second sets of signals.

19. The ultrasound system of claim 18, wherein analyzing the processed signals comprises identifying characteristics of the medium between the elongate flexible body and structure.

20. The ultrasound system of claim 18, wherein identifying characteristics comprises identifying movement of the medium.

21. The ultrasound system of claim 20, wherein the medium is blood and the structure is a blood vessel.

22. The ultrasound system of claim 18, wherein one or more processors are programed to isolate the response signal from an activated transducer having a lower frequency.

23. The ultrasound system of claim 18, further comprising a flexible expandable balloon arranged about the flexible body, wherein one or more of the plurality of transducers are arranged within the expandable balloon, and wherein calculating a plurality of distances between the elongate flexible body and an inner wall of the structure comprises calculating distances between the elongate flexible body and an inner wall of the expandable balloon.

24-32. (canceled)
